(19) United States
(12) Patent Application Publication
(10) Pub. No.: US 2023/0190253 A1
(43) Pub. Date: Jun. 22, 2023

(54) ANTERIOR CERVICAL DISC DISTRACTOR

(71) Applicant: BOARD OF REGENTS OF THE UNIVERSITY OF NEBRASKA, Lincoln, NE (US)

(72) Inventors: Joseph Howard McMordie, Omaha, NE (US); Daniel Surdell, Omaha, NE (US)

(21) Appl. No.: 18/068,208

(22) Filed: Dec. 19, 2022

Related U.S. Application Data
(60) Provisional application No. 63/291,089, filed on Dec. 17, 2021.

(51) Int. Cl. A61B 17/02

(52) U.S. Cl. CPC A61B 17/0206; A61B 2017/0256; A61B 2017/00473

(57) ABSTRACT

A medical device configured to engage a vertebrae body and a method of assembling a medical device. The device may include a head piece containing two distraction blades and a mechanism operable by the handle for driving apart/together the distraction blades. The device may further include an interface on the handle operable to engage and drive the mechanism of the head piece. The removable handle may include a grip portion separated from the interface by an elongate rod so that the handle, when attached to the head piece, is operable for insertion of the head piece into a corridor of a patient, and for driving the mechanism. With the medical device, a surgeon may operate to expose the disc space on at least one of the cervical, thoracic, or lumbar vertebrae.

### RELATED APPLICATION

The present application claims the benefit of co-pending U.S. Provisional Patent Application No. 63/291,089, filed Dec. 17, 2021, the entire contents of which is hereby incorporated by reference.

### FIELD

Embodiments described herein relate to medical instruments and, more particularly, to medical devices for use during anterior cervical discectomy, cervical arthroplasty, and other neurosurgical procedures of the spine.

### SUMMARY

Distraction devices are known for use during anterior cervical discectomy, cervical arthroplasty, and other neurosurgical procedures of the spine. However, it has been observed that the working corridor through the soft tissues of the neck may be crowded by the distraction devices that are available, thus limiting access to the cervical disc space and inhibiting maneuverability of other surgical instruments through the soft tissue corridor.

In some independent aspects, the invention may provide a low-profile distraction device with splayed distraction blades and a removable handle for adjusting the wedging action of the distraction blades. In some independent aspects, the invention may provide a method of using the distraction device in performing a surgery.

In some independent aspects, a medical device may be configured to engage a vertebrae body. The medical device may generally include a handle and a head configured to be removably coupled to the handle. The handle may include an elongate rod extending along a rod axis between a first end and a second end, and a grip coupled to the first end. The head is removably coupled to the second end. The head may include a frame defining an aperture, a pair of blades coupled to the frame for pivoting motion between a closed position and a splayed position, and a screw positioned in the aperture. The screw may be configured for movement along a central axis and includes a tip configured to engage the pair of blades. Movement of the screw moves the pair of blades between the closed position and the splayed position.

In some independent aspects, a medical device may include a handle extending along an axis; and a head configured to be removably coupled to the handle. The head may include a frame and a pair of blades pivotally coupled to the frame for movement between a closed position and a splayed position. The handle may be configured to couple to the head such that rotation of the handle about the axis drives the pair of blades between the closed position and the splayed position.

In some independent aspects, a method of operating a medical device configured to engage a vertebrae body may be provided. The medical device may include a handle extending along an axis and a head including a frame and a pair of blades pivotally coupled to the frame. The method may generally include removably coupling the handle and the head, and rotating the handle about the axis to move the pair of blades between a closed position and a splayed position.

Other independent aspects of the embodiments may become apparent by consideration of the detailed description, claims and accompanying drawings.

### BRIEF DESCRIPTION OF THE DRAWINGS

FIG. 1A schematically illustrates use of a conventional caspar pin distractor in a first stage of anterior cervical discectomy.

FIG. 1B schematically illustrates use of a conventional caspar pin distractor in a second stage of anterior cervical discectomy.

FIG. 2 schematically illustrates use of an anterior cervical disc distractor according to one embodiment of the present disclosure.

FIG. 3 is a side view of the anterior cervical disc distractor of FIG. 2.

FIG. 4 is a perspective first end view of the anterior cervical disc distractor of FIG. 2 illustrating a head separated from a handle.

FIG. 5 is a perspective second end view of the head of the anterior cervical disc distractor of FIG. 2.

FIG. 6 is a cross-sectional side view of the head of the anterior cervical disc distractor of FIG. 2, illustrated without the drive screw.

FIG. 7 is a cross-sectional side view of the handle of the anterior cervical disc distractor of FIG. 2.

FIG. 8A is a side view of the head of the anterior cervical disc distractor of FIG. 2, illustrated in a first (e.g., closed) position.

FIG. 8B is a side view of the head of the anterior cervical disc distractor of FIG. 2, illustrated in a second (e.g., intermediate) position.

FIG. 8C is a side view of the head of the anterior cervical disc distractor of FIG. 2, illustrated in a third (e.g., splayed) position.

FIG. 9 schematically illustrates a perspective view of the head of the anterior cervical disc distractor of FIG. 2, in use in an anterior cervical discectomy.

FIG. 10A illustrates an anterior cervical disc distractor of a second embodiment of the present disclosure.

FIG. 10B illustrates an anterior cervical disc distractor of a third embodiment of the present disclosure.

### DETAILED DESCRIPTION

Before any embodiments are explained in detail, it is to be understood that the embodiments described herein are provided as examples and the details of construction and the arrangement of the components described herein or illustrated in the accompanying drawings should not be considered limiting. Also, it is to be understood that the phraseology and terminology used herein is for the purpose of description and should not be regarded as limited.

The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting, and coupling. Further, “connected” and “coupled” are not restricted to physical or mechanical connections or couplings, and may include electrical connections or couplings, whether direct or indirect.

Relative terminology, such as, for example, “about”, “approximately”, “substantially”, etc., used in connection with a quantity or condition would be understood by those of ordinary skill to be inclusive of the stated value and has the meaning dictated by the context (for example, the term includes at least the degree of error associated with the measurement of, tolerances (e.g., manufacturing, assembly, use, etc.) associated with the particular value, etc.). Such terminology should also be considered as disclosing the range defined by the absolute values of the two endpoints. For example, the expression “from about 2 to about 4” also discloses the range “from 2 to 4”. The relative terminology may refer to plus or minus a percentage (e.g., 1%, 5%, 10% or more) of an indicated value.

Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way but may also be configured in ways that are not listed.

The embodiment(s) described below and illustrated in the figures are presented by way of example only and are not intended as a limitation upon the concepts and principles of the present disclosure. As such, it will be appreciated that variations and modifications to the elements and their configuration and/or arrangement exist within the spirit and scope of one or more independent aspects as described.

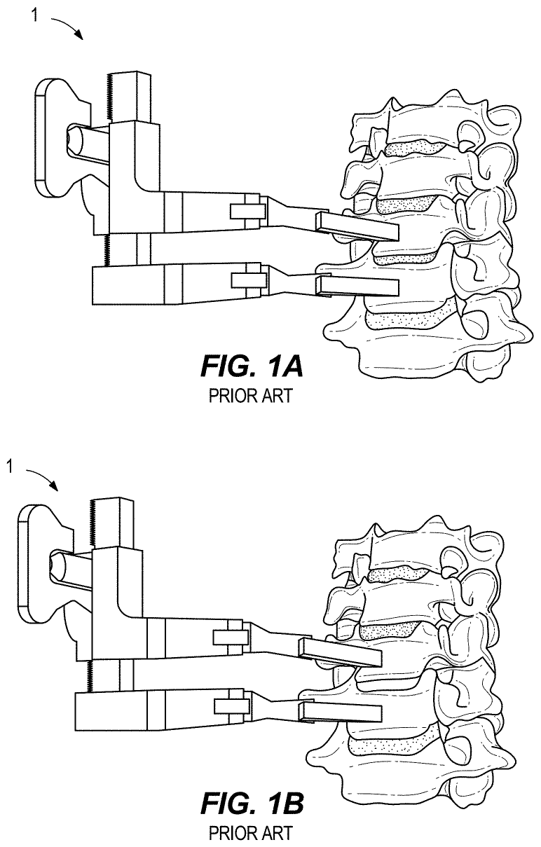

FIGS. 1A and 1B schematically illustrate use of a conventional caspar pin distractor 1 during anterior cervical discectomy. Two caspar pins anchor into the adjacent vertebrae on either side of the disc space. The caspar pins are connected to a linear ratchet that is operated to separate the adjacent vertebrae or, in other words, to distract open the disc space.

The caspar pin extension through the working corridor (the space allowing access through the anterior region to the vertebrae) is limiting to the surgeon while accessing the disc space. It is undesirable to increase the size of the working corridor, as increasing the size would result in additional damage to the patient and in longer recovery times. Additionally, when using caspar pin distracters, the distraction forces are limited to the anterior portion of the vertebral body where the caspar pins are placed. Deep soft tissue working corridors, particularly present in obese patients, those with kyphotic cervical spines, and those with inferiorly angled C6-7 and C7-T1 disc spaces, are difficult to access with the caspar distractor as the length of its distractor arms are limited.

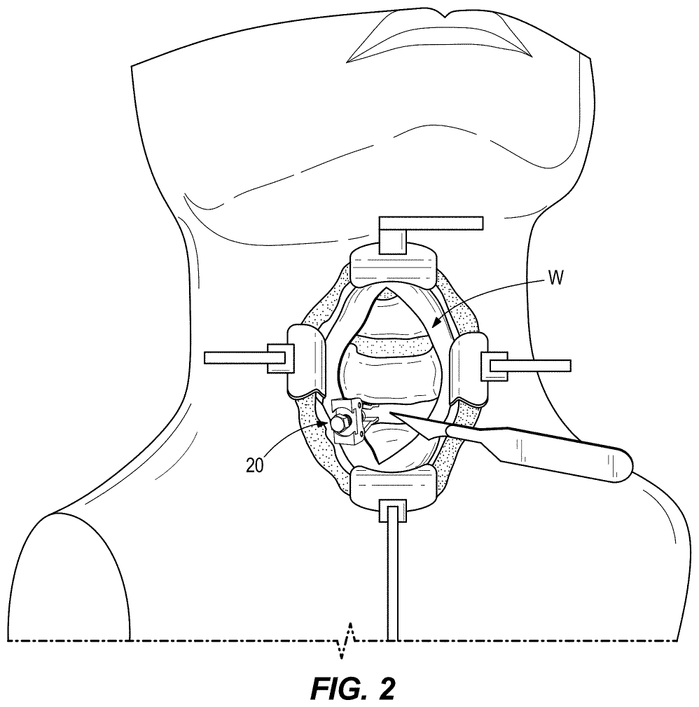

Described here with reference to the FIGS. 2-9 is one embodiment of a cervical disc spreader and distractor device 20 (also referred to herein as a medical device 20 or simply a device 20) which may improve access to the anterior cervical disc space during anterior cervical discectomy, cervical arthroplasty, and other neurosurgical procedures of the spine. With reference to FIG. 2, the illustrated device 20 provides a low profile and adjustable means to perform distraction for exposing the anterior cervical disc space from anterior to posterior without limiting the working corridor W in the region of interest.

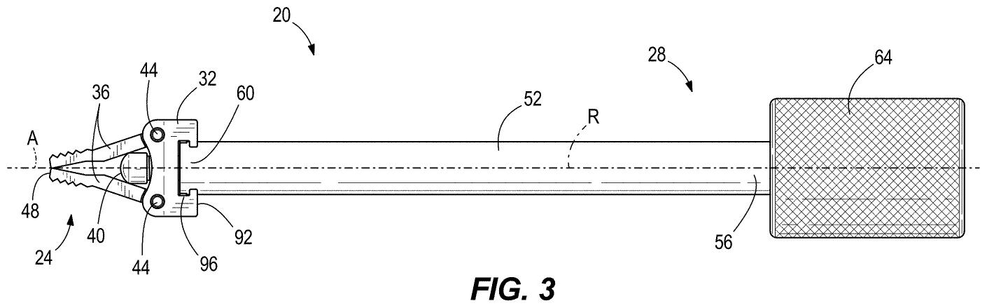

With reference to FIG. 3, the device 20 comprises a head 24 and a driver or handle 28 used to move and operate the head 24. In the illustrated construction, the head 24 and the handle 28 are removably connectable. The device 20 may be constructed with any suitable surgical grade material, including but not limited to grade stainless steel, nitinol, plastics, or other materials.

The illustrated head 24 includes a frame 32, a pair of distraction blades 36, and a driver or an actuator 40 (e.g., a screw, as illustrated, or other threaded member) supported by the frame 32 for movement to a plurality of positions along a central axis A. The distraction blades 36 are mounted to the frame 32 on either side of the axis A for pivoting movement by respective pivot joints (e.g., pins 44) so that the distraction blades 36 may be pivoted or splayed to a variety of positions in accordance with the position of the actuator 40. The pins 44 are positioned perpendicular to the axis A so that movement of the blades 36 moves tips 48 of the blades 36 toward or away from the axis A.

The handle 28 has a quick-connect coupling with the head 24 to facilitate removable connection of the handle 28 and the head 24. When connected, the handle 28 is operable to position the head 24. When rotated, the connected handle 28 acts as a driver to separate the distraction blades 36.

With continued reference to FIG. 3, the illustrated handle 28 includes an elongate rod 52 extending along a rod axis R between a first end 56 and an opposite second end 60, which is couplable to the head 24. The first end 56 of the handle 28 includes a grip 64. In the illustrated embodiment, the grip 64 has an enlarged sized compared to the elongate rod 52 (i.e., a diameter of the grip 64 is larger than a diameter of the elongate rod 52). The grip 64 may also be textured (e.g., knurled) and/or provided with an ergonomic shape for holding and rotating the handle 28. As shown in FIG. 7, the illustrated grip 64 is hollow to decrease the weight of the device 20 and thereby improve ergonomics.

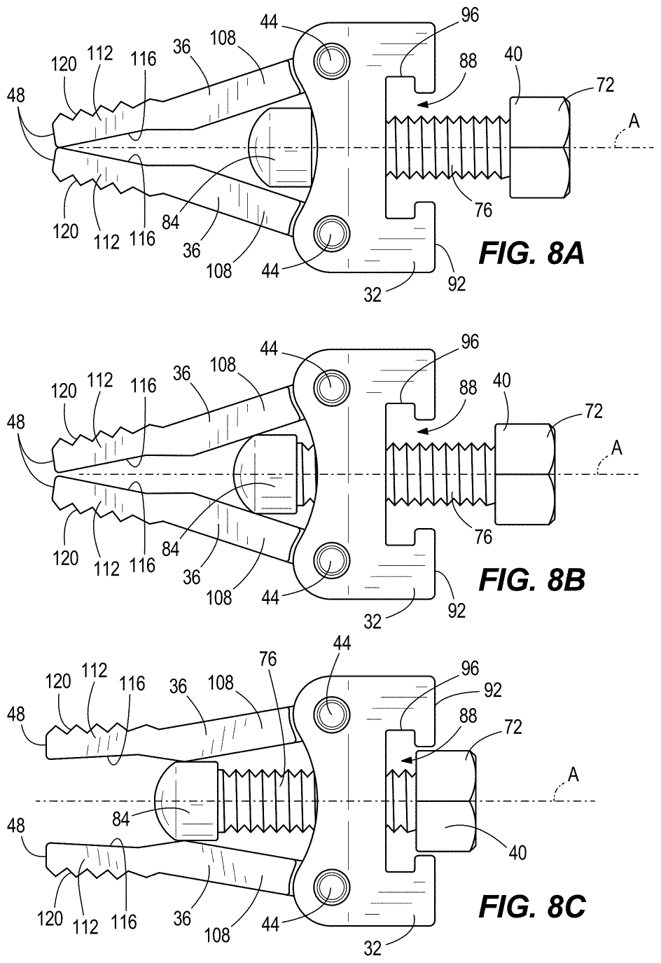

As mentioned above, in the illustrated embodiment, the handle 28 is removably coupled to the head 24. The handle 28 is selectively and alternatively connectable to the head 24 in two modes—in a first mode (see FIG. 3), the handle 28 acts as an insertion tool to allow proper placement and adjustment of the device 20 as needed, and, in a second mode, the handle 28 serves to drive the distraction blades 36 at least from a closed position (FIG. 8A) toward a splayed position (FIG. 8C).

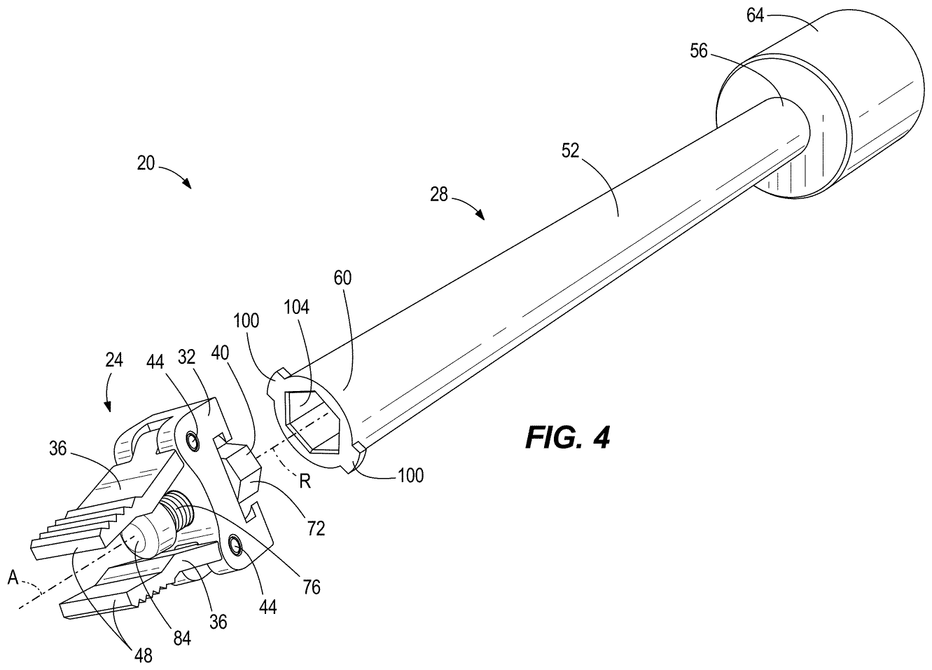

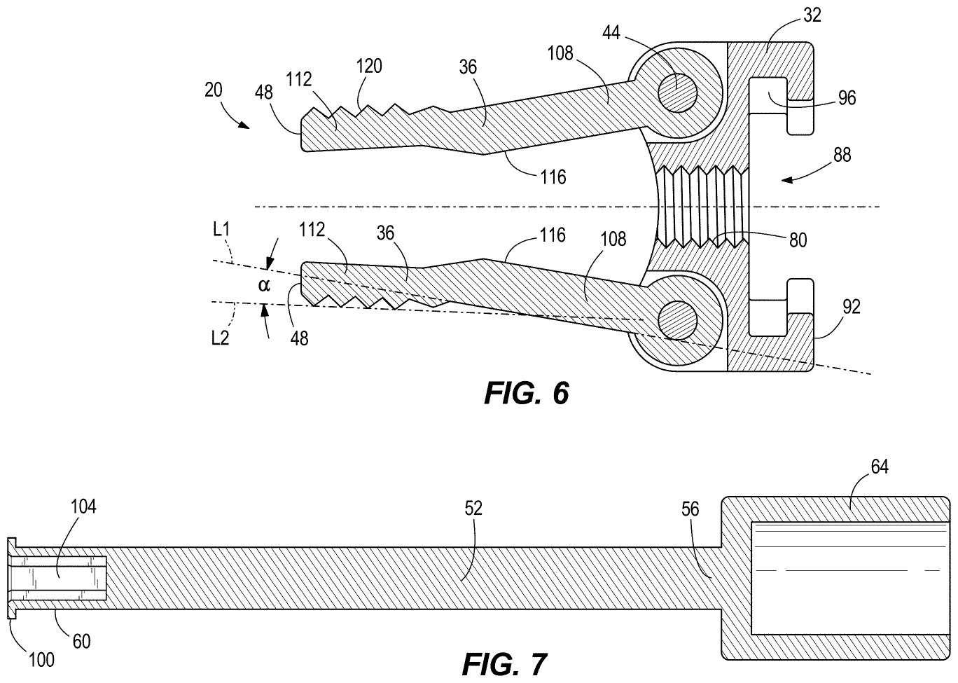

Turning to FIG. 4, the screw 40 has a screw head 72 on a rear side of the frame 32 and a shank 76 with a tip 84 on a forward side of the frame 32. The shank 76 is threaded, and the screw 40 is received within a threaded hole or aperture 80 (FIG. 6) in the frame 32. The threads of the shank 76 and the aperture 80 cooperate to cause linear displacement of the screw 40 relative to the frame 32 when the screw 40 is rotated about the axis A. The end or tip 84 of the shank 76 is configured to selectively contact or engage the distraction blades 36. In some embodiments (as shown), the tip 84 includes a separate cap coupled to the shank 76 and having cam surfaces that engage the blades 36.

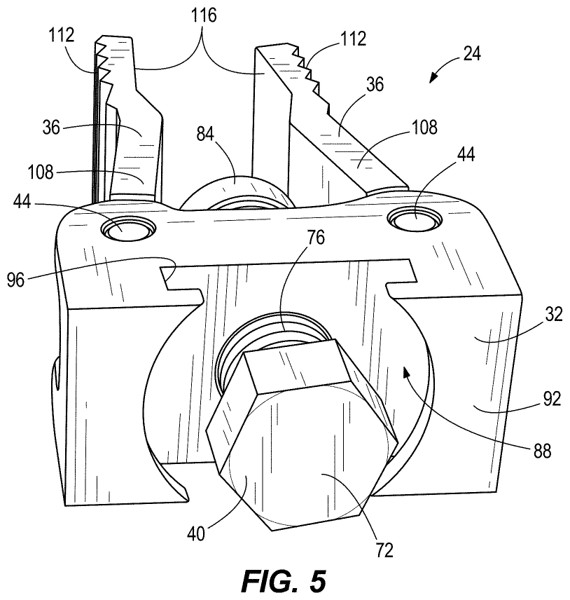

As shown in FIGS. 3-4, the second end 60 of the elongate rod 52 selectively engages the frame 32 of the head 24. With reference to FIG. 5, the illustrated frame 32 includes a slot 88 formed in the rear side. The slot 88 is recessed from the rear surface 92 and includes a groove 96 extending perpendicular to the axis A. The screw 40 may be coupled to the frame 32 with the screw head 72 adjacent the slot 88. The second end 60 of the handle 28 includes flanges or lugs 100, for example, a diametrically opposed pair of lugs 100, adapted to engage the groove 96 of the slot 88 and establish a coupling between the head 24 and the handle 28 in the second mode. The coupling allows placement of the head 24 by manipulation of the handle 28 alone.

The second end 60 of the handle 28 may be coupled to the frame 32 by a sequence of orientation and rotation, without tools or separate fasteners. For example, the handle 28 is positioned with the lugs 100 oriented toward open sides of the slot 88 while the handle 28 is moved axially along the axis A of the head 24 until the lugs 100 align with the groove 96. The handle 28 is subsequently pivoted about the axis A to move the lugs 100 into the groove 96 and lock the handle 28 to the head 24. In some embodiments, the handle 28 is pivoted about the axis A by about 90 degrees. In some embodiments, a different degree of pivoting movement may be used to engage the lug(s) 100 in the groove 96.

In some embodiments (not shown), the groove 96 may have a varying height (e.g., be tapered), and the lugs 100 are retained in the groove 96 by friction. In some embodiments (not shown), the groove 96 may include additional features to engage the lugs 100 and lock the handle 28 to the head 24. Other methods of retaining the lugs 100 within the groove 96 are also contemplated.

As shown best in FIG. 4, the second end 60 of the illustrated handle 28 also includes an interface for establishing a driving connection with the screw 40. Specifically, the second end 60 includes a socket 104 engageable with the screw head 72 of the screw 40. The illustrated socket 104 is constructed (e.g., has a shape (for example, square, hexagonal (as shown), non-circular, etc.) complementary to the shape of the screw head 72. When the screw head 72 of the screw 40 is received in the socket 104, rotation of the handle 28 is transmitted to the screw 40 such that the screw 40 displaces along the axis A due to the threaded engagement between the shank 76 and the aperture 80.

When the handle 28 is coupled to the head 24 in the first mode, the handle 28 may be positioned with the rod axis R in line with the axis A of the head 24 and with the lugs 100 overlying the rear surface 92. As the handle 28 is moved toward the frame 32, the screw head 72 is received by the socket 104, drivingly coupling the handle 28 and the screw 40. The handle 28 is then rotated about the axes A, R to rotate and linearly move the screw 40.

With reference to FIG. 6, the blades 36 extend between a proximal end 108 and a distal end 112 and include co-facing surfaces 116. At the proximal end 108 the co-facing surfaces 116 are engageable by the shank 76 of the screw 40, as described in further detail below. The distal ends 112 of the blades 36 are configured for vertebrae engagement on the opposed outer surfaces 120. The illustrated surfaces 120 include serrations to increase friction. In some embodiments, other forms of texturing may be used. As illustrated, each blade 36 does not extend along a single extension line; instead, the distal end 112 is beveled, splayed, or otherwise offset from the proximal end 108 to deviate from the path established by the proximal end 108.

As seen in FIG. 6, the proximal end 108 establishes an extension line L between its ends, which may or may not be followed directly by the co-facing surface 116 or an outer surface thereof. As illustrated, the extension line L1 converges toward the central axis A. In such an orientation, the distal end 112 extends from the proximal end 108 toward the tip 48 of the blade 36 along a path that is further from the central axis A than following the path established by the proximal end 108, as shown by the extension line L2.

As illustrated, the distal end 112 extends generally parallel to the central axis A. Regardless of the orientation of the blades 36 with respect to the axis A, the distal end 112 forms a non-zero angle α with the proximal end 108. In other words, in an operational configuration in which the proximal end 108 (and its surface 116) is converging toward the central axis A (as taken from proximal to distal), the distal end 112 converges toward the central axis A to a lesser degree (including not converging at all). The reduced convergence may be a reduced angle or, in the case of a basic offset, simply a reduced proximity as compared to following the path of the proximal end 108.

As shown in FIGS. 8A-8C, the screw 40 may be actuated to extend between the co-facing surfaces 116 of the distraction blades 36. The shank 76 of the screw 40, for example the tip 84, contacts the co-facing surfaces 116 to drive the distraction blades 36 apart when the screw 40 is rotated in an advancing direction (toward the distal end 112).

FIG. 8A illustrates an initial position, or a closed position, of the head 24 and the blades 36. In this position, the co-facing surfaces 116 at the proximal ends 108 are initially positioned to converge. The screw 40 is at an exemplary first position, with the screw head 72 spaced from the rear surface 92 of the frame 32 by a first amount, the tip 84 of the screw 40 adjacent to the front of the frame 32 and spaced from the distraction blades 36. In the initial position, the tips 48 of the blades 36 are in contact with each other. When the handle 28 is coupled to the screw 40 in the first mode, the lugs 100 are positioned behind or rearwardly of the rear surface 92 of the frame 32.

As shown in FIG. 8B, as the screw 40 is rotated relative to the frame 32, the tip 84 is moved in the advancing direction into contact with the co-facing surface 116 at the proximal end 108 of the blades 36. As shown in FIG. 8C, continued advancement of the screw 40 causes the screw 40 to drive the blades 36 from the closed position to a splayed position or open position. Due to the offset nature of the blades 36, the splayed position or open position of the blades 36 may be a maximum opening of the blades 36. In some embodiments, the open position may not be the maximum opening of the blades 36.

In the splayed or open position, the tips 48 are spaced from each other to define a working space therebetween. In this position, the screw head 72 is at a second distance from the rear surface 92 of the frame 32. In some embodiments (as shown), the screw head 72 is partially received by the slot 88. At least a portion of the screw head 72 extends rearwardly of the rear surface 92 of the frame 32 so that the socket 104 of the handle 28 may engage the screw head 72 without entering the slot 88 or engaging the groove 96.

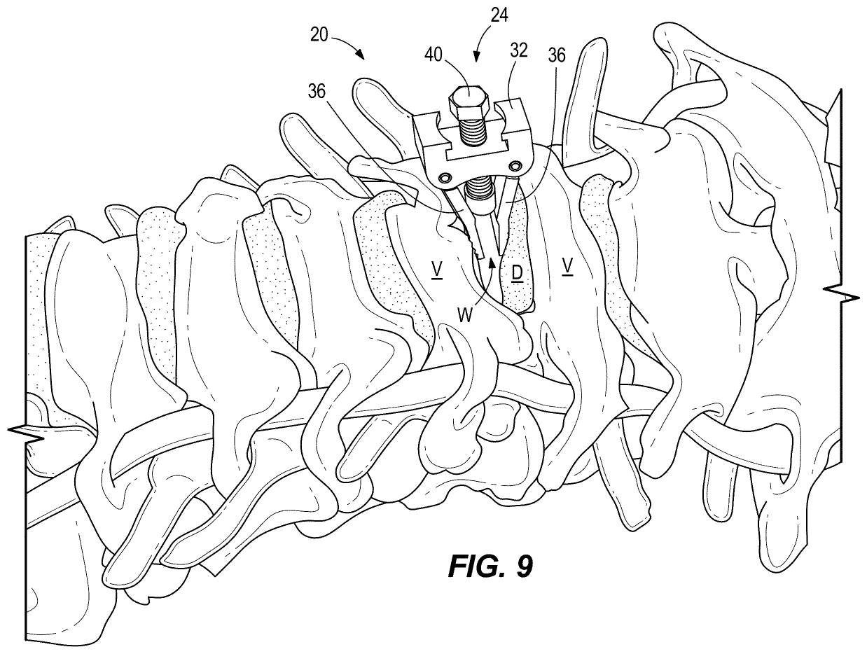

With reference to FIG. 9, the device 20 is illustrated in use. In operation, the handle 28 is connected to the head 24 in the second mode to couple the head 24 and handle 28 for movement together. Specifically, the lugs 100 are positioned in the grooves 96 to releasably lock the handle 28 relative to the head 24. The head 24 is therefore carried with the handle 28, and, once the region of interest has been exposed, as shown in FIG. 2, the handle 28 is used to position the distraction blades 36 between adjacent vertebrae body within or adjacent a disc space.

The handle 28 may then be disconnected from the head 24. The handle 28 is removed by rotating the handle 28 about the axis A relative to the head 24 until the lugs 100 are disengaged from the groove 96 and removed from the slot 88. The handle 28 is then connected to the head 24 in the first mode to act as a driver to separate the distraction blades 36 via the screw 40. In some embodiments, the handle 28 may engage an alternate wedge mechanism to distract the blades 36.

In the illustrated embodiment, the handle 28 is connected to the head 24 to rotationally couple the screw 40 and the handle 28. The handle 28 is aligned with the head 24 and positioned so the socket 104 receives the screw head 72. The screw 40 receives rotational input from the handle 28 and acts to wedge apart the distraction blades 36. Thus, the handle 28 is rotated about the axis A in order to advance the screw 40, as shown in the change of position in FIGS. 8A-8C.

The linear displacement of the screw 40 moves the tip 84 into engagement with the co-facing surfaces 116 of the blades 36, drives distraction of the blades 36 and, as a result, widens the space between adjacent vertebrae, allowing access to the disc space. The distal ends 112 of the distraction blades 36 are angled with respect to the proximal ends 108, as described above, so that, when the blades 36 are in the splayed position, a working space is provided between the blades 36 for performing medical operations, such as a discectomy. Once the vertebrae are distracted, the handle 28 is detached from the head 24 by axially moving the handle 28 rearwardly. The handle 28 is removed from the surgical space, leaving the working corridor free during any subsequent operations.

Once the operation is completed for that region of interest, the handle 28 is re-attached onto the head 24 in the first mode, by engaging the screw 40 with the second end 60 of the handle 28. The screw head 72 is positioned within the socket 104 to couple the handle 28 and the screw 40 for corotation. The handle 28 is then used to retract the screw 40 to allow the distraction blades 36 to move inwardly toward the axis A. In some embodiments, as illustrated, the blades 36 are moved toward the axis A by the restorative force of the adjacent vertebrae returning to position. In some embodiments, the distraction blades 36 may be biased to retract toward the axis A (e.g., by a biasing member, such as a spring).

The handle 28 may be re-connected to the head 24 in the second mode, by positioning the lugs 100 within the slot 88 and rotating the handle 28 to lock the lugs 100 into place in the groove 96. The handle 28 is then manipulated to remove the device 20 from the patient.

The device 20 does not require pin placement or violation of the bone. The adjacent level of vertebrae is not at risk and the soft tissues of the neck are not retracted with the present anterior cervical disc space spreader device 20 as the device 20 provides distraction focally at the intended disc space. This device 20 may be inserted into the disc space and may be advanced to the posterior limit of the disc space to ensure equal distraction from the anterior to posterior disc margin avoiding a narrowed posterior corridor and providing access to posterior osteophytes and views of the spinal cord. Deep soft tissue corridors are not limiting to this device 20 as the device 20 sits entirely on the anterior cervical spine and does not extend out of the incision.

The illustrated device 20 provides a low-profile means of anterior cervical disc space distraction compared to current methods of caspar pin extension. With the conventional caspar pin distractor 1, the working corridor through the soft tissues of the neck is crowded by the distraction devices. This device 20 may improve access to the cervical disc space while maintaining maneuverability of other surgical instruments through the soft tissue corridor. This device 20 may be scaled for use to distract any vertebrae pair, including the cervical, thoracic, and lumbar vertebrae, for accessing the disc space therebetween.

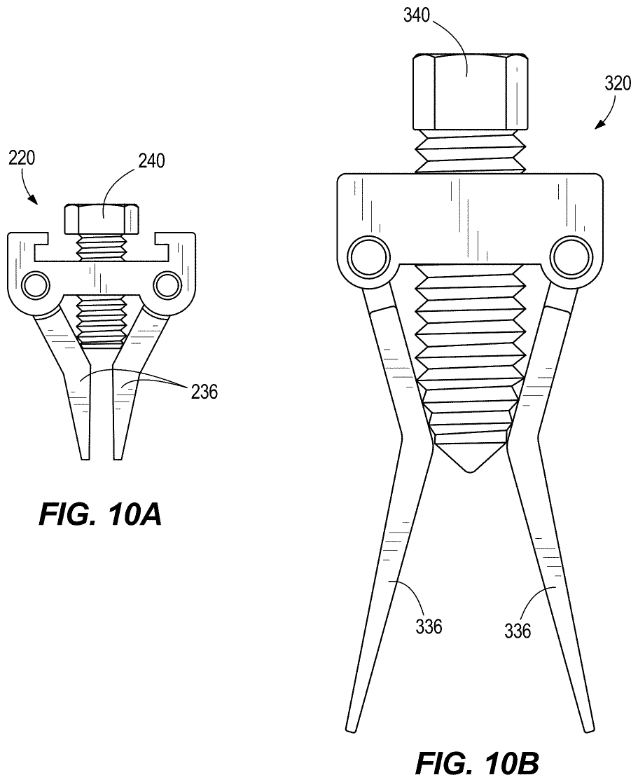

FIGS. 10A and 10B illustrate additional embodiments of low-profile disc distractors 220, 320. As shown in the figures, the size and shape of the blades 236, 336 are varied to provide optimal disc distraction. Additionally, the relative size and shape of the screws 240, 340 are varied to provide optimal driving of the blades 236, 336. Other modifications to the design are also possible without departing from the intended scope of the present disclosure.

One or more independent features and/or independent advantages of the embodiments may be set forth in the following claims:

What is claimed is:

1. A medical device configured to engage a vertebrae body, the medical device comprising:
a handle including an elongate rod extending along a rod axis between a first end and a second end, and a grip coupled to the first end; and
a head configured to be removably coupled to the second end of the handle, the head including
a frame defining an aperture,
a pair of blades coupled to the frame for pivoting movement between a closed position and a splayed position, and
a screw positioned in the aperture and configured for movement along a central axis, the screw having a tip configured to engage the pair of blades and, during movement of the screw, to move the pair of blades between the closed position and the splayed position.

2. The medical device of claim 1, wherein the elongate rod is configured to drivingly engage the screw.

3. The medical device of claim 2, wherein the elongate rod includes a socket, and the screw includes a screw head, and wherein the screw head is receivable by the socket to drivingly couple the handle and the screw such that rotation of the handle about the rod axis rotates the screw, moving the screw linearly along the central axis.

4. The medical device of claim 3, wherein the socket is hexagonally shaped.

5. The medical device of claim 1, wherein the grip includes a knurled surface.

6. The medical device of claim 1, wherein a diameter of the grip is larger than a diameter of the elongate rod.

7. The medical device of claim 1, wherein each blade of the pair of blades has a serrated surface configured to engage the vertebrae body.

8. The medical device of claim 1, wherein each blade includes a proximal end and a distal end, and wherein the proximal end extends along an extension line at an angle to an extension line of the distal end.

9. The medical device of claim 1, wherein the handle is configured to engage the frame to position the head adjacent the vertebrae body.

10. The medical device of claim 1, wherein the second end of the elongate rod includes a lug, and wherein the frame includes a slot, and wherein the lug is configured to engage the slot to couple the head for movement with the handle.

11. The medical device of claim 10, wherein the second end of the elongate rod includes a lug, and wherein the frame includes a slot, wherein the lug is engageable with the slot by rotating the handle relative to the central axis.

12. A medical device configured to engage a vertebrae body, the medical device comprising:
a handle extending along an axis; and
a head configured to be removably coupled to the handle, the head including a frame and a pair of blades pivotally coupled to the frame for movement between a closed position and a splayed position;
wherein the handle is configured to couple to the head such that rotation of the handle about the axis drives the pair of blades between the closed position and the splayed position.

13. The medical device of claim 12, wherein the handle is configured to be coupled to the head in a first mode, in which rotation of the handle moves the pair of blades between the closed position and the splayed position, and a second mode, in which movement of the handle moves the head relative to the vertebrae body.

14. The medical device of claim 13, wherein the head further including a driver configured to move the pair of blades between the closed position and the splayed position, and wherein, in the first mode, the handle engages the driver.

15. The medical device of claim 14, wherein the driver includes a threaded member received within a threaded aperture in the frame and configured to engage the handle such that rotation of the handle causes linear displacement of the threaded member.

16. The medical device of claim 15, wherein the threaded member includes a tip engageable with the pair of blades to move the pair of blades from the closed position toward the splayed position.

17. The medical device of claim 12, the head further including a driver configured to move the pair of blades between the closed position and the splayed position, wherein the handle is configured to selectively and alternatively engage the frame to move the head relative to the vertebrae body or the driver to move the pair of blades.

18. The medical device of claim 12, wherein the head is configured to be positioned between adjacent vertebrae, and wherein movement of the pair of blades to the splayed position drives the adjacent vertebrae further apart.

19. The medical device of claim 18, wherein, once the head is positioned between the adjacent vertebrae in the splayed position, the handle is removable from the head.

20. A method of operating a medical device configured to engage a vertebrae body, the medical device including a handle extending along an axis, and a head including a frame and a pair of blades pivotally coupled to the frame, the method comprising:
removably coupling the handle and the head; and
rotating the handle about the axis to move the pair of blades between a closed position and a splayed position.
